(19) United States
(12) Patent Application Publication
(10) Pub. No.: US 2022/0015935 A1
(43) Pub. Date: Jan. 20, 2022

(54) ORTHOSIS AND METHOD FOR CONTROLLING AN ORTHOSIS

(71) Applicant: Ottobock SE & Co. KGAA, Duderstadt (DE)

(72) Inventors: David HOCHMANN, Steinfurt (DE); Heiko DREWITZ, Gleichen (DE); Gordon SIEWERT, Duderstadt (DE); André Müller, Immingerode (DE); Olaf KROLL-ORYWAHL, Northeim (DE)

(73) Assignee: Ottobock SE & Co. KGAA, Duderstadt (DE)

(21) Appl. No.: 17/312,381

(22) PCT Filed: Dec. 9, 2019
(86) PCT No.: PCT/EP2019/084225

§ 371 Date: Jun. 9, 2021

(30) Foreign Application Priority Data
Dec. 12, 2018 (DE) 10 2018 131 852.9

(51) Int. Cl. A61F 5/01

(52) U.S. Cl. CPC A61F 5/0111; A61F 2005/0188; A61F 2005/0155

(57) ABSTRACT

The invention relates to an orthosis for a lower limb, with a foot part, which has a sole for supporting a foot, and with at least one leg rail, which extends from the foot part and, in the fitted state, extends along a lower leg and has devices for supporting and/or securing the leg rail on the lower leg, wherein the orthosis is assigned at least one sensor for detecting orthosis parameters, which sensor is coupled to a control device, and the foot part is assigned at least one device coupled to the control device and/or materials for changing the stiffness of the sole.

The invention relates to an orthosis for a lower limb, having a foot part which has a sole for supporting a foot, and having at least one leg rail which extends from said foot part and which in the fitted state extends along a lower leg and which has devices for supporting and/or fastening the leg rail on the lower leg. The invention likewise relates to a method for controlling such an orthosis.

Orthoses are function-ensuring medical aids which lie against the body and which are used to assist body parts with restricted functional capability, wherein, in particular, stabilizing, mobilizing, load-relieving and corrective functions of the orthosis are of primary concern. So-called AFOs (ankle-foot orthoses) are orthoses which are used to compensate insufficient lower leg musculature following peripheral paralysis and to ensure knee stability in the case of quadricep weakness.

Insufficient lower leg musculature is nowadays treated almost exclusively by way of so-called foot lifting orthoses which have no influence on the knee joint or the calf musculature and therefore cannot prevent the occurrence of incorrect positioning as a tertiary consequence of an impairment. Quadricep weakness is treated predominantly using orthoses which also extend across the knee, so-called KAFOs (knee-ankle-foot orthoses).

DE 10 2004 030 570 A1 describes a support element for a lower leg orthosis, in the case of which a lower-leg sleeve is connected by means of the elastically deformable support element to a foot sleeve. The lower-leg sleeve and the foot sleeve can be fastened to the lower leg and to the foot respectively by way of hook and loop fasteners. An electrical circuit with an electromechanical transducer in the form of a piezo element converts mechanical energy during the deformation of the support element into an electrical signal that is stored in a memory. A resetting or damping element is provided by means of which the spring rate of the support element can be varied in a manner dependent on a signal. The stored electrical energy is converted into mechanical energy again as required, for example in order to vary the spring rate of the support spring, in particular in order to stiffen the support element.

WO 2005/025446 A2 describes an orthosis with a sole and with a lower leg part. From the heel region of the sole to a toe region of the sole, different thicknesses and stiffnesses are provided in order to provide improved support of the foot.

It is an object of the present invention to provide an orthosis and a method for controlling an orthosis, with which an adaptation to changes in the usage conditions is possible in order to increase the wearing comfort and the benefit for the patient.

According to the invention, said object is achieved by means of an orthosis having the features of the main claim and a method having the features of the coordinate claim. Advantageous embodiments and refinements of the invention are disclosed in the subclaims, in the description and in the figures.

The orthosis according to the invention for a lower limb, having a foot part which has a sole for supporting a foot, and having at least one leg rail which extends from said foot part and which in the fitted state extends along a lower leg and which has devices for supporting and/or fastening the leg rail on the lower leg, provides that the orthosis is assigned at least one sensor for detecting orthosis parameters, which at least one sensor is coupled to a control device, and that the foot part is assigned at least one device, which is coupled to the control device, and/or materials for varying the stiffness of the sole. By means of the detection of orthosis parameters by means of sensors and the conversion of the orthosis parameters in a control device into signals with which the stiffness of the sole is varied, and individual adaptation of the sole to the ambient conditions, to the patient and to their gait behavior is possible. In particular, the stiffness of the heel lever and/or the stiffness of the forefoot lever can be varied. It is thus possible to provide a dynamically adaptive foot orthosis for patients with muscle weakness, which foot orthosis can adapt to changes in the ambient conditions and to the individual circumstances of an orthosis user. The adaptation furthermore results in a knee-stabilizing action, whereby the patient can walk in an improved manner and more safely.

The leg rail may be assigned at least one device, which is coupled to the control device, for varying the stiffness of the leg rail, such that, aside from the influencing of the stiffness of the forefoot lever and/or of the heel lever, the stiffness of the leg rail can also be varied, such that the stiffness of the leg rail can be actively varied for example in a manner dependent on the angle of inclination of the sole, the forces that occur upon heel strike, or on the basis of the ascertainment of position data of a knee joint axis.

The device for varying the stiffness in the sole may for example have an actuator for displacing a sole insert, a piezo element, a magnetorheological medium, a preloaded spring element, and/or a thermal element, by means of which, individually or in a combination of one or more of the above-stated elements with one another, the desired adaptation and adaptability of the stiffness of the sole to the respective walking situation or to a patient can be implemented.

The change in the stiffness in the leg rail may be implemented for example by means of a displacement of a leg rail insert, a piezo element, a magnetorheological medium, a preloaded spring element and/or a thermal element, by means of which, individually or in a combination of one or more of the above-stated elements with one another, the desired adaptation and adaptability of the stiffness of the leg rail to the respective walking situation or to a patient can be implemented.

A joint may be arranged between the leg rail and the foot part, which joint is assigned a brake and/or a damper device which is coupled to the control device and which is activated or deactivated in a manner dependent on the detected orthosis parameters. In this way, aside from an individual adaptation of the leg rail by means of an orientation in the joint and possibly an adaptation of the stiffness of the leg rail, the resistance moment about the joint can additionally be varied.

The leg rail may be manufactured as a separate component and arranged articulatedly on the foot part, such that retroactive installation in the joint region is possible. As an alternative to this, the leg rail and the foot part may be formed as a single piece, wherein an articulated embodiment may be implemented by means of a targeted material weakening and the formation of a so-called film hinge.

In the case of an articulated arrangement on the foot part, the leg rail may be assigned a drive for setting the orientation of the leg rail relative to the foot part and/or may be assigned a fixing device for fixing the orientation relative to the foot part. In this way, it is possible for the position of the lower leg relative to the sole to be individually set and fixed or else varied, such that a physiologically correct orientation is always realized, for example when standing.

The sensors may be arranged in the forefoot region and/or in the heel region of the sole, on the leg rail, or at the transition from the leg rail to the foot part, in order to detect relevant orthosis parameters. Likewise, at least one sensor may be arranged outside the orthosis, for example in order to detect the position of the knee axis and transmit this to the control device.

The sensors may be in the form of force sensors, pressure sensors, acceleration sensors, gyroscopes, position sensors and/or angle sensors.

It is possible for multiple devices and/or materials to be provided for varying the stiffness of the sole and/or of the leg rail in mutually different orientations, in order to allow both the sole and the leg rail to be configured to be settable individually and in accordance with the situation.

The sole is advantageously designed for accommodating the entire foot in order, in the case of the typical indications for a lower leg orthosis, to be able to provide complete support of the foot such that the patient can be provided with optimum treatment.

The method according to the invention for controlling an orthosis, as has been described above, provides that the stiffness of the sole and/or of the leg rail is varied in a manner dependent on sensor values that have been ascertained by means of the sensors arranged on the orthosis. In this way, an adaptation to ambient conditions is possible. A negative change in gait behavior and in the sequences and states that are correct in terms of physiology and motion can be avoided through the wearing of an optimized and adaptively adaptable orthosis.

One aspect of the method provides that a starting value for a center of gravity position of the force action points on the sole and/or an orientation of the leg rail or of the sole in space is specified and, if the starting value is overshot by a specified amount, a variation of the stiffness of the sole and/or of the leg rail is performed in a manner dependent on the position, ascertained by means of the sensors, of the force action point and/or on the orientation of the sole and/or of the leg rail in space.

In addition or alternatively to this, the orientation of the leg rail relative to the foot part may be varied in a manner dependent on the sensor values, such that an orientation of the leg rail and thus of the lower leg that stabilizes the knee and relieves the knee of load can be achieved.

In the case of a joint being arranged between the leg rail and the foot part, the rotational resistance thereof can be varied in a manner dependent on the detected orthosis parameters, whereby an adaptation to the ambient conditions or changes in physiological status can also be compensated and taken into consideration.

An exemplary embodiment of the invention will be discussed in more detail below on the basis of the figures. The same reference designations are used to denote identical components. In the figures:

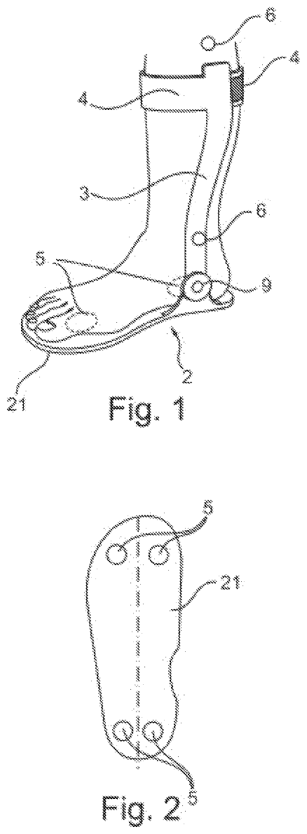

FIG. 1 shows a schematic view of a fitted orthosis;

FIG. 2 shows a view of an orthosis from below;

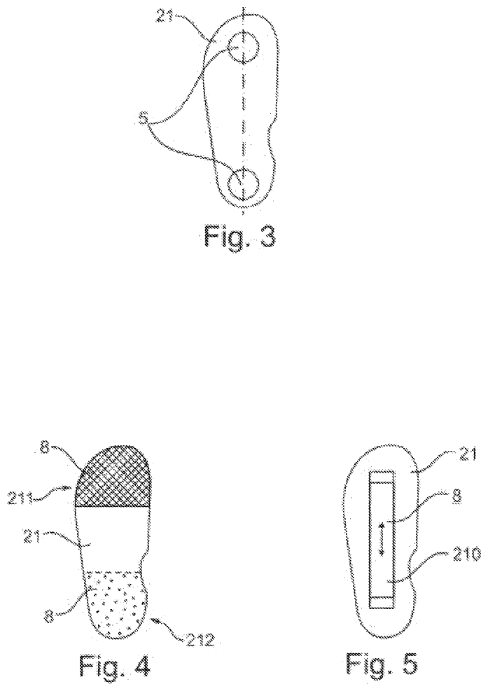

FIG. 3 shows a variant of the sole;

FIG. 4 shows a sole configuration with regions of different stiffness;

FIG. 5 shows a view of a sole with a displaceable sole insert;

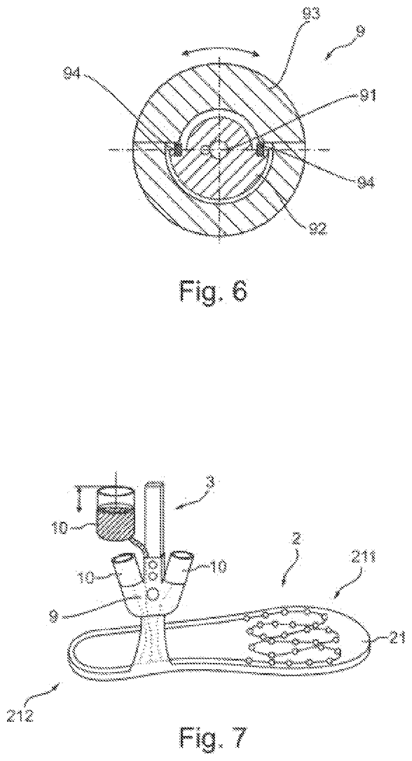

FIG. 6 shows a sectional illustration through a joint;

FIG. 7 shows a variant of the invention with a hydraulics hose in the sole;

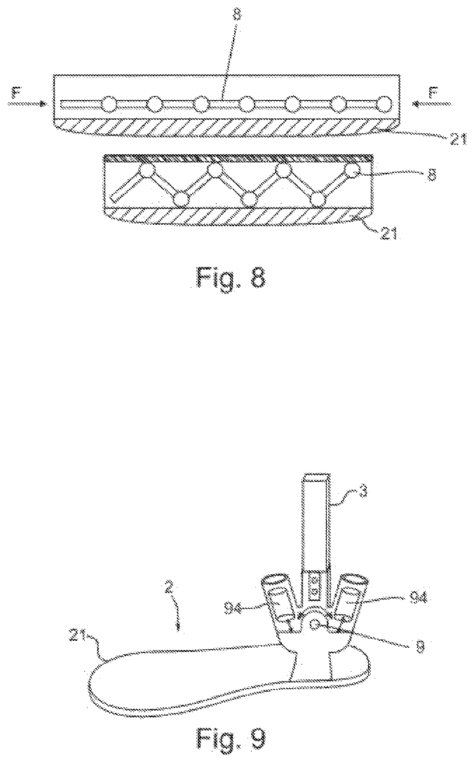

FIG. 8 shows a schematic sectional illustration of a stiffening element;

FIG. 9 shows an orthosis with ankle joint and dampers;

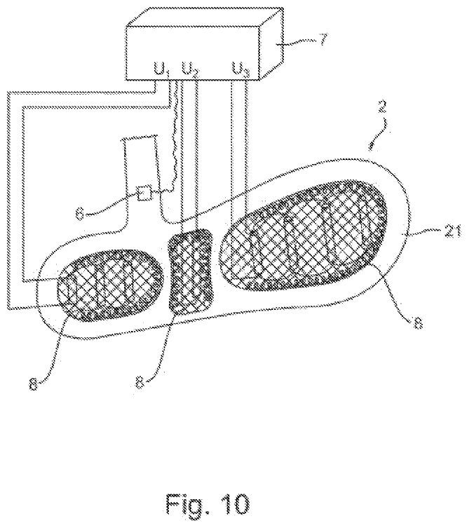

FIG. 10 shows a sole with thermal elements;

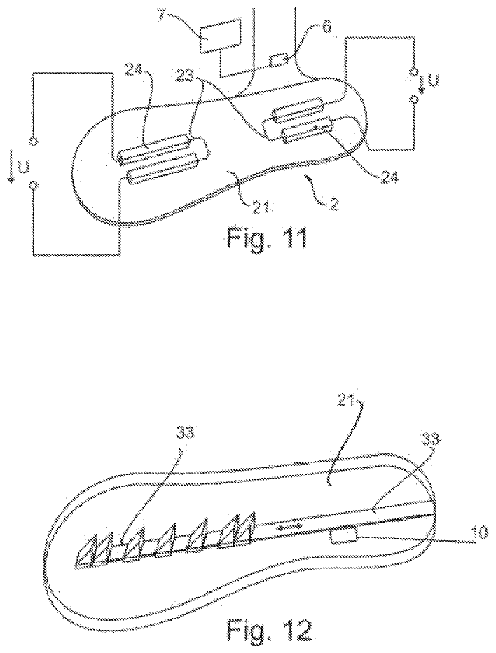

FIG. 11 shows a sole with magnetorheological fluids and electromagnets;

FIG. 12 shows a sole with strips of different elasticities which are displaceable relative to one another;

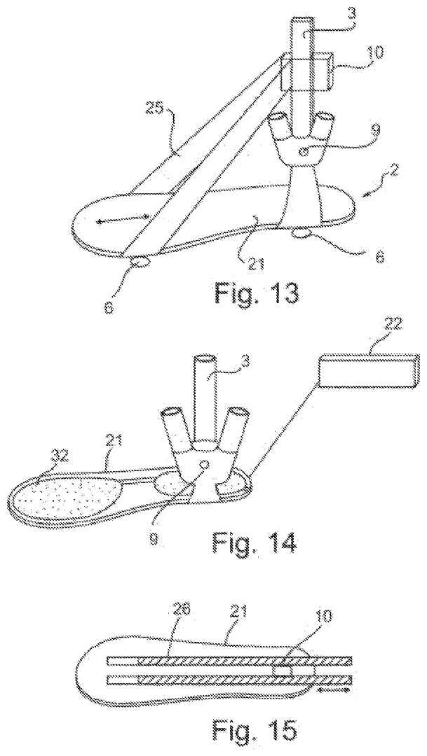

FIG. 13 shows an orthosis with an actuator on a lower leg rail and with displaceably mounted stiffening elements in the sole;

FIG. 14 shows a sole with evacuable chambers;

FIG. 15 shows a sole with displaceable spring elements;

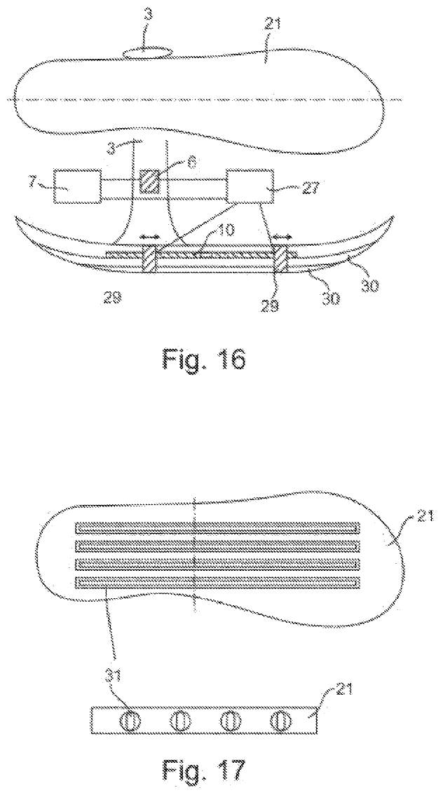

FIG. 16 shows a sole with displaceable leaf springs arranged one above the other;

FIG. 17 shows a variant with rotary bars; and

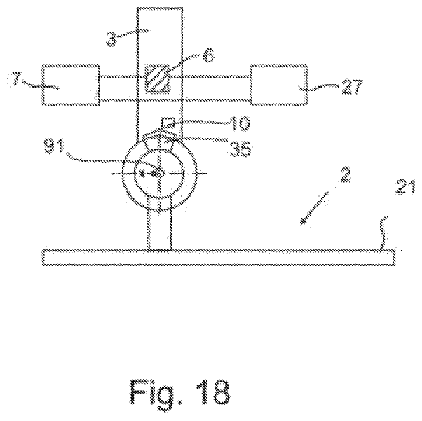

FIG. 18 shows an illustration of an orthosis with a brake.

FIG. 1 illustrates an orthosis 1 for a lower limb, having a foot part 2, in a schematic illustration. The foot part 2 has a sole 21 on which a foot can be supported over a full area. On the foot part 2, there is arranged a leg rail 3 which has a device 4 in the form of a shin stirrup and a hook and loop fastener for fastening to a lower leg. The leg rail 3 is connected by means of a joint 9 to the foot part 2. A setting of the orientation of the leg rail 3 relative to the foot part 2 is possible by means of the joint 9.

In the foot part 2, the leg rail 3 and/or the devices 4 for supporting the leg rail 3 on the lower leg, there may be arranged sensors 6 which detect orthosis parameters. It is possible for only one sensor 6 overall to be provided, it is likewise possible for one sensor 6 to be arranged in each of the various components of the orthosis, and it is likewise possible for multiple sensors 6 to be provided in or on one or more components. Furthermore, a sensor 6 may be provided as a position sensor for the position of a compromise axis, which sensor is attached to a patient outside the orthosis 1. Angle sensors for the angle in the upper ankle joint, pressure sensors for measuring the pressure load in the tibia structure, pressure sensors in the sole 21, acceleration sensors for detecting gait behavior or underlying surface conditions, and piezoelectric sensors for detecting ground contact or heel pushoff, may be provided.

FIG. 2 illustrates a view of the foot part 2, with the sole 21, from below. The sole 21 extends over the entire length of a foot and has, to both sides of a longitudinal axis, pressure sensors 5 which record pressures or forces on the sole 21.

FIG. 3 illustrates a variant of FIG. 2 in which the sensors 5 in the sole 21 are arranged on the longitudinal axis, such that only two sensors 5 or sensor regions are provided in the sole 21.

In FIG. 4, the sole 21 is divided into different sole regions, specifically into a heel region 212 and a toe region 211 which is separated from said heel region by a central region. In the heel region 212 and in the toe region 211, there are arranged materials which change their mechanical characteristics as a result of the application of external fields, for example electrical voltage, or through the activation of magnetic fields. Through the application of external fields to said regions 211, 212, the strength and stiffness of the respective sole regions 211, 212 can be varied. In this way, it is possible to vary the effective heel lever and the effective forefoot lever. The heel lever, in the static state, prevents falling backward and, in the dynamic state, that is to say during walking, can influence the knee flexion. The forefoot lever prevents falling forward when standing and can assist the pushoff and influence the knee extension during walking. The softer the sole 21 in the respective lever, the shorter the respective effective lever, and the stiffer the respective region of the sole 21, the longer the effective lever.

FIG. 5 shows a variant of the invention in which, in the sole 21, there is provided a displaceable sole insert 210 as a device 8 for varying the stiffness of the sole 21. The sole insert 210 can, in a manner dependent on the ascertained orthosis parameters, be displaced in the direction of the toe region 211 or of the heel region 212, whereby the heel lever and the forefoot lever respectively change.

A further variation of the orthosis is possible by means of variation of the construction of the orthosis by virtue of the orientation of the leg rail 3 relative to the sole 21 being varied. By means of the angle between the leg rail 3 and the sole 21, the load on the knee joint when standing and when walking is influenced, such that it is possible to achieve a position, which preserves the knee and which is expedient with regard to gait physiology, of the leg rail 3 and thus of the lower leg relative to the sole 21 and thus relative to the foot seated thereon over a full area.

It is furthermore possible to vary the stiffness of the leg rail 3 by means of devices which correspond to the devices 8 for varying the stiffness of the sole. It is likewise possible for a brake or an adjusting device to be assigned to the leg rail 3, such that, in the joint 9, an adjustment of the angle of the leg rail 3 with respect to the sole 21 can be performed. The adjustment may be performed in a manner dependent on the orthosis parameters ascertained by means of the sensors 5, 6.

FIG. 6 shows a detail illustration of a joint 9 without foot part 2 and leg rail 3. In the exemplary embodiment illustrated, a joint outer part 93 is provided with a recess in which a joint inner part 92 is arranged. In the exemplary embodiment illustrated, the joint outer part 93 is coupled to the leg rail 3 (not illustrated). The joint inner part 92 is coupled to the foot part 2 (not illustrated), for example to the proximal end of a foot section that extends in a proximal direction from the foot part 2 or from the sole 21. The joint outer part 93 is mounted so as to be pivotable about a pivot axis 91, which is positioned for example at the height of the natural ankle joint. The pivotability is illustrated by the double arrow. Both on the joint outer part 93 and on the joint inner part 92, there are formed projections and stops which are situated opposite one another. Between the stops, there are arranged damper elements 94, which may be in the form of rubber dampers and which, aside from a damping action, also impart a resetting force counter to a pivoting movement, such that the joint outer part 93 is moved back into an initial position. Aside from an embodiment with stops as projections or stop surfaces, a design with equivalent action can also be achieved by means of a non-circular embodiment either of the recess in the joint outer part 93 or in the outer contour of the joint inner part 92, with correspondingly arranged damper elements 94.

Instead of resiliently elastic damper elements, damper devices 94 may be installed in the joint 9 or assigned to said joint 9, which damper devices are coupled to a control device 7 (not illustrated). The damper device 94 can then be set with regard to its resistance to a pivoting movement. The damper device 94 may be in the form of a hydraulic system which, at the respective contact points or force transmission points between the joint outer part 93 at the joint inner part 92, has chambers or pistons via which hydraulic fluid is conducted from one chamber into the other chamber. Between these chambers, there may be arranged a valve or a settable throttle which is coupled to the control device 7 and which provides a variable resistance in a manner dependent on sensor data. By means of the control device 7, the damper device 94 can be activated or deactivated or set with regard to the desired damping. As control parameters or orthosis parameters, there may be provided, for example, load sensors, spatial position sensors or angle sensors which detect an angle between the leg rail 3 and the foot part.

FIG. 7 shows a variant of the invention in which the leg rail 3 is fastened to the foot part 2.

In the foot part 2, a hose system is embedded in or fastened to the sole 21. The sole 21 has flexibility, which can be varied through variation of the degree of filling of the hose system. On the leg rail 3, there is arranged an actuator 10 which, by means of a piston, pumps hydraulic fluid into, or draws hydraulic fluid out of, the hydraulic line. Depending on the degree of filling of the hydraulic line, the sole 21 stiffens or becomes flexible. It can be seen in FIG. 7 that a longer section of hydraulic line is laid in the forefoot region or toe region 211 than in the heel region 212. In this way, it is possible to generate different stiffnesses in the forefoot region 211 and in the heel region 212. By means of the actuator 10, the hose system may also be filled with a compressible fluid, for example air, rather than with an incompressible fluid.

Instead of or in addition to the actuator 10 arranged on the leg rail 3, it is possible for two actuators 10 to be arranged on the joint body 9, which actuators act as pumps. By means of pistons, which can be driven by the pivoting movement of the leg rail 3 relative to the foot part 2, the pistons can be moved, and pressure can correspondingly be built up or depleted in the line system in the sole 21 by means of either a hydraulic fluid or a pneumatic fluid. Depending on the filling pressure, the sole 21 stiffens or becomes more flexible.

FIG. 8 schematically shows a further variant of the invention. The sectional view shows two states of the device 8 for varying the stiffness; in the upper illustration, the device 8 is guided in the form of a link chain, in an extended state, in a channel of the sole 21. In this state, the sole 21 is relatively soft and flexible. In the lower illustration of FIG. 8, the link chain 8 has been collapsed, such that, within the channel, it lies against the upper and lower boundaries and thus imparts a stiffening action. The greater the force with which the link chain is collapsed, the stiffer the sole 21.

FIG. 9 shows a further variant of the invention. The construction corresponds substantially to the construction as per FIG. 7, but instead of a piston or an actuator in the joint body, two dampers 94 are arranged in the joint body, which dampers are designed to be settable, in particular in a manner dependent on sensor data that have been ascertained by sensors (not illustrated). The dampers 94 counteract a pivoting about the pivot axis and preferably simultaneously provide a resetting force in order to move the joint 9 back into an initial position when there are no longer any external forces acting. The dampers 94 may be in the form of mechanical dampers, for example elastomer elements, hydraulic dampers, pneumatic dampers or magnetorheological dampers, and influences the pivoting movement of the foot part 2 relative to the leg rail 3. The sole 21 may, in addition to the damper devices 94 in the joint body, be provided with one or more devices for varying the stiffness.

FIG. 10 shows, in a variant of the invention, the sole 21 of the foot part 2 with thermal elements as devices 8 for varying the stiffness of the sole 21. The thermal elements 8 are integrated in the sole 21 and act on a material insert or on the sole material, which material insert or sole material varies its strength and elasticity in a manner dependent on the temperature. The warmer the material becomes, the softer and more flexible the material insert and thus also the sole 21 in its entirety become. By means of the control device 7, in a manner dependent on sensor values of the sensor 6, different voltages are conducted, or electrical current is conducted for different lengths of time, through resistance elements in order to cause warming. The inserts composed of a material which exhibits different stiffnesses depending on the prevailing temperature are, in the exemplary embodiment illustrated, arranged in three regions, in the forefoot region, in the heel region and in the midfoot region. All regions can be controlled separately by the control device 7 in order to be able to set different stiffnesses independently of one another. Aside from warming, it is basically also possible to achieve an increase in stiffness, and a variation in flexibility, by way of cooling.

A further variant of the invention is shown in FIG. 11, in which, instead of material inserts composed of a material with temperature-dependent stiffness, lines with magnetorheological fluid 23 are integrated in the sole 21. By means of the control device 7, in a manner dependent on data from a sensor 6, a magnetic field is generated by means of electromagnets 24, such that the particles in the magnetorheological fluid 23 are varied with regard to their viscosity. In this way, it is possible for the stiffness in the sole 21 to be influenced very quickly through variation of the magnetic field.

A further variant of the invention is shown in FIG. 12, in which, in the sole 21, two strips 33 with different elastic and stiff regions are arranged one above the other, which strips are mounted so as to be displaceable relative to one another. By means of an actuator 9, it is possible for the two strips 33 to be displaced back and forth in the longitudinal extent, as indicated by the double arrow. On the strips 33, there may be arranged wedge-like projections which can be pushed one over the other or pushed one inside the other in order to form differently elastic or flexible regions. If the wedges are moved apart, an intermediate space opens between the wedges and allows increased flexibility, and if said wedges are pushed together, the stiffness of the sole 21 can be increased.

FIG. 13 shows a further variant of the orthosis with an actuator 10 which is arranged on the leg rail and which is adjusted in a manner dependent on data from sensors 6 that is incorporated in the sole 21. The actuator 10 can vary the length of straps or tension elements 25, for example by winding-up and unwinding, or be displaced along the longitudinal extent of the sole 21, as indicated by the double arrow, in order to thus displace stiffening elements, displace or rotate disks or strips 33 with different elastic characteristics with respect to one another, or in order to increase a preload in the straps 25, in order to thus influence the stiffness of the sole 21 in a manner dependent on sensor data from the sensors 6.

A further variant of the invention is illustrated in FIG. 14, in which, in the sole 21, there are arranged air chambers 32 that can be filled with a granulate. By means of a vacuum pump 22, air can be drawn out of the air chambers, whereby the granulate is compacted and an increase in strength and thus a stiffness of the sole 21 in the respective region is realized.

A further variant of the invention is illustrated in FIG. 15, in which, within the sole 2, there are arranged spring steel strips 26 which are arranged so as to be displaceable in the longitudinal extent of the sole 21. The strips 26 are displaced by means of an actuator 10 in or on the sole 21 and thus allow a variation of the stiffness of the sole in the region in which the strips 26 are not present. Aside from an embodiment of the strips 26 composed of spring steel, these may also be formed from some other material.

A further variant of the invention is shown in FIG. 16, in which the upper illustration shows a view from below and the lower illustration shows a schematic sectional illustration. The control device 7 is coupled to a sensor 6, and the sensor 6 may be in the form of a spatial position sensor or inertial measurement unit (IMU). An energy store 27, for example a battery, provides electrical energy in order to move a screw drive as actuator 10. By means of clamping devices 29, leaf springs 30 that are arranged one above the other are coupled to the screw drive 10. The screw drive 10 is in two parts, such that a variation of the stiffness is possible for the forefoot and the heel separately by displacement of the individual leaf springs 30 relative to one another.

FIG. 17 shows a further variant of the invention with a rotary bars 31 arranged of the sole 21. The rotary bars 31 are arranged in bores or recesses within the sole 21 and can be rotated by means of an actuator 10 (not illustrated). In the exemplary embodiment illustrated, the rotary bars 31 are of oval form or formed so as to be flattened on both sides, resulting in different geometrical moments of inertia. If, as shown in the lower illustration of FIG. 17, the rotary bars 31 are positioned with their longer cross section upright, that is to say in a vertical direction, the stiffness is increased owing to the greater effective geometrical moment of inertia. If the rotary bars 31 are rotated through 90°, the stiffness of the sole 21 is reduced owing to the greater flexibility of the rotary bars 31 within the sole 21.

FIG. 18 shows a variant of the invention in which the pivoting movement of the foot part 2 relative to the leg rail 3 about the pivot axis 91 can be influenced by means of a brake 35. In the exemplary embodiment illustrated, the brake device 35 is, in the form of a disk brake; it may be in the form of a shoe-type brake, wrap spring brake, electrically acting brake or some other brake device. By means of a sensor 6 that it coupled to a control unit 7, energy for activating or deactivating the actuator 10 is provided by means of energy stores 27, and the brake device 35 is then activated or deactivated by means of said actuator. In this way, it is possible for the movement and mobility between the leg rail 3 and the foot part 2 to be influenced in a manner dependent on the orthosis parameters detected by means of the sensors 6. It is furthermore provided that the brake device 35 is in the form of a motor that can be deactivated in order to maintain a selected orientation between leg rail 3 and foot part 2. The drive 35 may vary the orientation of the leg rail 3 with respect to the sole 21 in a manner dependent on sensor data, and at the same time serve as a brake or as a fixing device by virtue of the motor being deactivated and impeding or preventing the movement. The drive 35 may be coupled by means of a self-locking gearing to the foot part 2 such that, when the drive 35 is inactive, no relative displacement is possible.

1. An orthosis for a lower limb, having a foot part which has a sole for supporting a foot, and having at least one leg rail which extends from said foot part and which in a fitted state extends along a lower leg and which has devices for supporting and/or fastening the leg rail on the lower leg, characterized in that the orthosis is assigned at least one sensor for detecting orthosis parameters, wherein the at least one sensor is coupled to a control device, and in that the foot part is assigned at least one device, which is coupled to the control device, and/or materials for varying the stiffness of the sole.

2. The orthosis of claim 1, wherein the leg rail is assigned at least one device, which is coupled to the control device, for varying the stiffness of the leg rail.

3. The orthosis of claim 1, wherein the device for varying the stiffness of the sole has at least one of the following elements, or combinations thereof: an actuator for displacing a sole insert, a piezo element, a magnetorheological medium, a preloaded spring element, and/or a thermal element.

4. The orthosis of claim 1, wherein a joint is arranged between the leg rail and the foot part, and the joint is assigned a brake and/or a damper device which is coupled to the control device and which is activated or deactivated in a manner dependent on the detected orthosis parameters.

5. The orthosis of claim 1, wherein the leg rail is formed as a separate component and is arranged in articulated fashion on the foot part.

6. The orthosis of claim 5, wherein the leg rail is assigned a drive for setting the orientation relative to the foot part and/or is assigned a fixing device for fixing the orientation relative to the foot part.

7. The orthosis of claim 1, wherein sensors are arranged in the forefoot region and/or in the heel region of the sole, on the leg rail or at the transition from the leg rail to the foot part.

8. The orthosis of claim 1, wherein at least one sensor is arranged outside the orthosis.

9. The orthosis of claim 1, wherein the sensors are in the form of force sensors, pressure sensors, acceleration sensors, gyroscopes and/or angle sensors.

10. The orthosis of claim 1, wherein multiple devices and/or materials are provided for varying the stiffness of the sole and/or leg rail in mutually different orientations.

11. The orthosis of claim 1, wherein the sole is designed for accommodating an entire foot.

12. A method for controlling an orthosis of claim 1, wherein the stiffness of the sole and/or of the leg rail is varied in a manner dependent on sensor values that have been ascertained by means of the sensors (5, 6) arranged on the orthosis.

13. The method of claim 12, wherein a starting value for a center of gravity position of the force action points on the sole and/or an orientation of the leg rail or of the sole in space is specified and, if the starting value is overshot by a specified amount, a variation of the stiffness of the sole and/or of the leg rail is performed in a manner dependent on the position, ascertained by means of the sensors (5, 6), of the force action point and/or on the orientation of the sole and/or of the leg rail in space.

14. The method of claim 12, wherein the orientation of the leg rail relative to the foot part is varied in a manner dependent on the sensor values.

15. The method of claim 12, wherein, between the foot part and the leg rail, there is arranged a joint, the rotational resistance of which is varied in a manner dependent on the detected orthosis parameters.

16. An orthosis for a lower limb, the orthosis having:
a foot part which has a sole for supporting a foot, and
at least one leg rail which extends from said foot part and which in a fitted state extends along a lower leg and which has devices for supporting and/or fastening the leg rail on the lower leg;
wherein the orthosis further includes at least one sensor for detecting orthosis parameters, the at least one sensor being coupled to a control device;
wherein the at least one leg rail is assigned at least one device coupled to the control device for varying the stiffness of the leg rail, and wherein the leg rail is assigned a drive for setting the orientation relative to the foot part and/or is assigned a fixing device for fixing the orientation relative to the foot part; and
wherein the foot part is assigned at least one device which is coupled to the control device, and/or materials for varying the stiffness of the sole.

17. The orthosis of claim 16, wherein the device for varying the stiffness of the sole has at least one of the following elements, or combinations thereof: an actuator for displacing a sole insert, a piezo element, a magnetorheological medium, a preloaded spring element, and/or a thermal element.

18. The orthosis of claim 16, wherein a joint is arranged between the leg rail and the foot part, and the joint is assigned a brake and/or a damper device which is coupled to the control device and which is activated or deactivated in a manner dependent on the detected orthosis parameters.

19. The orthosis of claim 1, wherein the leg rail is formed as a separate component and is arranged in articulated fashion on the foot part.

20. An orthosis for a lower limb, the orthosis having:
a foot part which has a sole for supporting a foot, and
at least one leg rail which extends from said foot part and which in a fitted state extends along a lower leg and which has devices for supporting and/or fastening the leg rail on the lower leg;
wherein the orthosis further includes at least one sensor for detecting orthosis parameters, the at least one sensor being coupled to a control device;
wherein the at least one leg rail is assigned at least one device coupled to the control device for varying the stiffness of the leg rail; and
wherein the foot part is assigned at least one device coupled to the control device for varying the stiffness of the sole, the device for varying the stiffness of the sole including at least one of the following elements, or combinations thereof: an actuator for displacing a sole insert, a piezo element, a magnetorheological medium, a preloaded spring element, and/or a thermal element.
